(19) United States
(12) Patent Application Publication
(10) Pub. No.: US 2007/0123934 A1
(43) Pub. Date: May 31, 2007

(54) Delivery system for patent foramen ovale closure device

(76) Inventors: Brian K. Whisenant, Salt Lake City, UT (US); Clark C. Davis, Holladay, UT (US); Daryl R. Edmiston, Draper, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/534,996

(22) Filed: Sep. 25, 2006

Related U.S. Application Data
(60) Provisional application No. 60/720,911, filed on Sep. 26, 2005.

(51) Int. Cl. A61B 17/08

(52) U.S. Cl. 606/213

(57) ABSTRACT

A medical system for use in reducing the size of an internal tissue opening is disclosed. The medical system can include a patent foramen ovale or PFO closure device and a delivery device therefore. The PFO closure device can include left and right anchors connected by a stem. The delivery device is configured to selectively deploy at least one of the right or left anchors of PFO closure device so as to enable a practitioner to adjust PFO closure device during the positioning procedure. In one embodiment, the delivery device includes a filament operating shaft linked to the right anchor by one or more filaments. A practitioner can move the filament operating shaft to selectively deploy and retract the right anchor during positioning of the PFO closure device. Furthermore, the delivery device comprises a tether shaft for coupling delivery device to PFO closure device. In one embodiment, the tether shaft comprises a first portion and a flexible second portion, wherein the flexible second portion is at a distal end of the tether shaft.

### CROSS-REFERENCE TO RELATED APPLICATIONS

This application claims benefits from U.S. Provisional Patent Application, Ser. No. 60/720,911, filed on Sep. 26, 2005, entitled “DELIVERY SYSTEM FOR PFO CLOSURE DEVICE”, the disclosure of which is hereby incorporated by reference.

### BACKGROUND OF THE INVENTION

1. The Field of the Invention

The present invention relates generally to implanting medical devices within a patient. More particularly, the present invention relates to the field of patent foramen ovale (“PFO”) closure devices and delivery devices therefore.

2. The Relevant Technology

FIGS. 1A-1C depict various views of a heart. Heart 10 is shown in a cross-section view in FIG. 1A. In a normal heart, the right atrium 30 receives systemic venous blood from the superior vena cava 15 and the inferior vena cava 25 and then delivers the blood via the tricuspid valve 35 to the right ventricle 60. However, in heart 10, there is a septal defect between right atrium 30 and left atrium 40 of a patient's heart which is referred to as a patent foramen ovale (“PFO”). PFO is a birth defect that occurs when an opening between the upper two chambers of the heart fail to close after birth to a lesser or greater degree. This birth defect is sometimes also known as a “hole in the heart.”

Other problems with this condition are that a blood clot may travel freely between the left or right atria of the heart, and end up on the arterial side. This could allow the clot to travel to the brain, or other organs, and cause embolization, or even a heart attack. These and other similar defects (septal or otherwise), where some tissue needs to be closed to function properly include the general categories of atrial septal defects (“ASDs”), ventricular septal defects (“VSCs”) and patent ductus arterosus (“PDA”), and so forth.

The PFO, which is an open flap on the septum between the heart's right and left atria, is generally identified at 50. In a normal heart, left atrium 40 receives oxygenated blood from the lungs via pulmonary arteries 75 and then delivers the blood to the left ventricle 80 via the bicuspid valve 45. However, in heart 10 some systemic venous blood also passes from right atrium 30 through PFO 50, mixes with the oxygenated blood in left atrium 40 and then is routed to the body from left ventricle 80 via aorta 85.

During fetal development of the heart, the interventricular septum 70 divides right ventricle 60 and left ventricle 80. In contrast, the atrium is only partially partitioned into right and left chambers during normal fetal development as there is a foramen ovale. When the septum primum 52 incompletely fuses with the septum secundum 54 of the atrial wall, the result is a PFO, such as the PFO 50 shown in FIGS. 1A-1C, or an atrial septal defect referred to as an ASD.

FIG. 1C provides a view of the crescent-shaped, overhanging configuration of the typical septum secundum 54 from within right atrium 30. Septum secundum 54 is defined by its inferior aspect 55, corresponding with the solid line in FIG. 1C, and its superior aspect 53, which is its attachment location to septum primum 52 as represented by the phantom line. Septum secundum 54 and septum primum 52 blend together at the ends of septum secundum 54; these anterior and posterior ends are referred to herein as “merger points” and are respectively identified at 56a and 56p. The length of the overhang of septum secundum 54, the distance between superior aspect 53 and inferior aspect 55, increases towards the center portion of the septum secundum 54 as shown.

A tunnel 58 is defined by portions of septum primum 52 and septum secundum 54 between the merger points 56a and 56p which have failed to fuse. The tunnel 58 is often at the apex of the septum secundum 54 as shown. When viewed within right atrium 30, the portion of septum secundum 54 to the left of tunnel 58, which is referred to herein as the posterior portion 57p of the septum secundum 54, is longer than the portion of the septum secundum 54 to the right of tunnel 58, which is referred to herein as the anterior portion 57a of the septum secundum 54. In addition to being typically longer, the left portion also typically has a more gradual taper than the right portion, as shown. The area defined by the overhang of the anterior portion 57a of septum secundum 54 and the septum primum 52 and extending from the anterior merger point 56a toward tunnel 58 is an anterior pocket 59a. Similarly, the area defined by the overhang of the posterior portion 57p of septum secundum 54 and the septum primum 52 and extending from the posterior merger point 56p toward tunnel 58 is a posterior pocket 59p.

Conventional treatments for PFO (and related conditions), have generally involved invasive surgery, which presents a different, new set of risks to a patient. Although there are some less invasive treatments for PFO, these have typically been less efficient at closing the PFO opening than techniques involving invasive surgery. Accordingly, there is a continuing need for improved methods and devices for closing the PFO opening. In particular, there is a need for improved methods and devices for deploying PFO closure anchors in a patient.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1A is a cross-sectional view of a heart;

FIG. 1B is an enlarged cross-section view of the septum primum and the septum secundum and a PFO tunnel between the septum primum and the septum secundum;

FIG. 1C is a perspective view of the septum secundum with the tunnel and the septum primum shown in phantom;

FIG. 2 is a perspective view of a medical system including a PFO closure device and a delivery device therefore;

FIG. 3A is a partial break-away, perspective view of the delivery device of FIG. 2;

FIG. 3B is a partial break-away, perspective view of a portion of the medical system of FIG. 2;

FIG. 4A is a partial cross-sectional view of the delivery device of FIG. 2;

FIG. 4B is a partial cross-sectional view of the delivery device of FIG. 4 in a linked orientation;

FIG. 5A is a cross-sectional view of the medical system of FIG. 2 as it is being positioned in a PFO;

FIG. 5B is a cross-sectional view of the medical system of FIG. 2 as left anchor is deployed;

FIG. 5C is a cross-sectional view of the medical system of FIG. 2 illustrating deployment of left and right anchors;

FIG. 5D is a perspective view of left anchors as viewed from the left atrium;

FIG. 6A is a cross-sectional view of the PFO closure device as positioned in the PFO; and

FIG. 6B is a perspective view of the right anchor as viewed from the right atrium.

### BRIEF SUMMARY OF THE DISCLOSURE

The invention relates to a medical system for use in reducing the size of an internal tissue opening, such as a PFO. In one embodiment, the medical system can include a PFO closure device and a delivery device therefore. The PFO closure device can include left and right anchors connected by a stem. The proximal end of the stem can include a set of internal threads for use in selectively connecting and disconnecting the delivery device to the PFO closure device. The delivery device can enable a practitioner to efficiently secure the PFO closure device in an internal tissue opening. Furthermore, the delivery device can enable a practitioner to adjust and reposition the PFO closure device after left and right anchors are deployed by selectively retracting the right anchor. The ability to reposition the PFO closure device provides practitioners with the added ability of more effectively reducing the size of an internal tissue opening by being able to adjust the PFO closure device during insertion to achieve the best position.

In one embodiment, the delivery device can include a tether shaft for removably coupling the delivery device to the PFO closure device. The tether shaft can include a first rigid portion and a second flexible portion, wherein the flexible second portion can be coupled to the stem of the PFO closure device through a threaded arrangement. Furthermore, one or more filaments can be coupled to a movable filament operating shaft and looped around one or more arms of the right anchor. Movement of the filament operating shaft can cause movement of the right anchor between a deployed and retracted position. Thus, a practitioner can move the filament operating shaft with respect to the PFO closure device to selectively deploy and/or retract the right anchor of the PFO closure device.

To facilitate disconnecting the delivery device and the PFO closure device, the tether shaft can be selectively linked to the filament operating shaft. In one embodiment, the tether shaft can be rotatably coupled to a shuttle block and the filament operating shaft can be coupled to a filament drive rod. The filament drive rod can be linked to the shuttle block by two rod pins, each positioned in the filament drive rod on opposing side of the shuttle block. As the filament drive rod moves in a distal direction, one of the rod pins contacts and engages the shuttle block, thus causing the shuttle block to move within a housing. However, prior to the rod pin contacting and engaging the shuttle block, movement of the filament drive rod in the distal direction can cause the one or more filaments to slacken, thus deploying the right anchors.

Linking of the tether shaft to the filament operating shaft can be facilitated by the configuration of the housing. The housing can be configured such that as the shuttle block moves in the distal direction, a pin, which can be located in an aperture or opening in the shuttle block, can be forced into a recess in the filament drive rod. As such, movement of the pin into the recess of the filament drive rod links the tether shaft to the filament operating shaft via the shuttle block. In this manner, a practitioner can safely move the tether shaft and the filament operating shaft in concert without concern that the right anchor will be inadvertently retracted by the delivery device.

These and other objects and features of the present invention will become more fully apparent from the following description and appended claims, or may be learned by the practice of the invention as set forth hereinafter.

### DETAILED DESCRIPTION OF THE INVENTION

The present invention extends to systems, methods, and apparatus for deploying a device that can be suitable for reducing the size of an internal tissue opening. By way of explanation, the devices disclosed herein can be used for any internal tissue opening, although frequent reference is made herein to closing a PFO opening of a heart tissue using right atrial anchors and left atrial anchors for purposes of simplicity. Accordingly, it will be understood that references to PFO openings are not limiting of the invention.

In the following description, numerous specific details are set forth in order to provide a thorough understanding of the present invention. It will be obvious, however, to one skilled in the art that the present invention may be practiced without these specific details. In other instances, well-known aspects of PFO closure devices or medical devices in general have not been described in particular detail in order to avoid unnecessarily obscuring the present invention. In addition, it is understood that the drawings are diagrammatic and schematic representations of certain embodiments of the invention, and are not limiting of the present invention nor are they necessarily drawn to scale.

The invention extends to systems, methods, and apparatus for deploying a device that can be suitable for reducing the size of an internal tissue opening. An apparatus for reducing the size of an internal tissue opening, such as a PFO closure device, can include a left atrial anchor and a right atrial anchor. The PFO closure device can be deployed by use of a delivery device. In one embodiment, the delivery device can be removably coupled to the PFO closure device by a threaded arrangement. In addition to being selectively removable from the PFO closure device, the delivery device can be configured to enable a practitioner to adjust and selectively manipulate the right anchor of the PFO closure device.

In one embodiment, the delivery device can include a tether shaft for removably coupling the delivery device to the PFO closure device and a housing enclosing at least a portion of the tether shaft. The tether shaft can include a first rigid portion and a second flexible portion, wherein the flexible second portion can be coupled to the stem of the PFO closure device through a threaded arrangement. However, the tether shaft can also be configured to have substantially the same rigidity along its length. Furthermore, the delivery device can be connected to the PFO closure device by one or more filaments. The filaments can be linked to the right and/or left anchor(s) such that movement of the filaments can cause movement of the respective anchor. In this manner, the anchor(s) of the PFO closure device can be selectively deployed and thereafter adjusted by a practitioner.

In one embodiment, the tether shaft can be selectively linked to the filaments to facilitate disconnecting the delivery device from the PFO closure device. For example, the tether shaft can be coupled to a movable member, such as a shuttle block, which in turn can be linked to a filament member, such as a filament drive rod or a filament operating shaft. The filament member can be coupled to the housing and can be linked to the shuttle block by a movable pin within the shuttle block. In one embodiment, the pin can move into an aperture or recess in the filament member, thus linking the filament member to the shuttle block. In this manner, the tether shaft can be linked to the filaments, such that movement of the filament member in a distal direction causes movement of the tether shaft in the distal direction.

The configuration the housing can facilitate linking of the tether shaft to the filament operating shaft. The housing can be configured such that as the shuttle block moves in the distal direction, a pin, which can be located in an aperture or opening in the shuttle block, can be forced into a recess in the filament drive rod. In this manner, the filament drive rod links the tether shaft to the filaments via the filament operating shaft, the pin and the shuttle block. In this manner, a practitioner can safely move the tether shaft and the filament operating shaft in concert with a reduced concern that the right anchor will be inadvertently retracted by the delivery device.

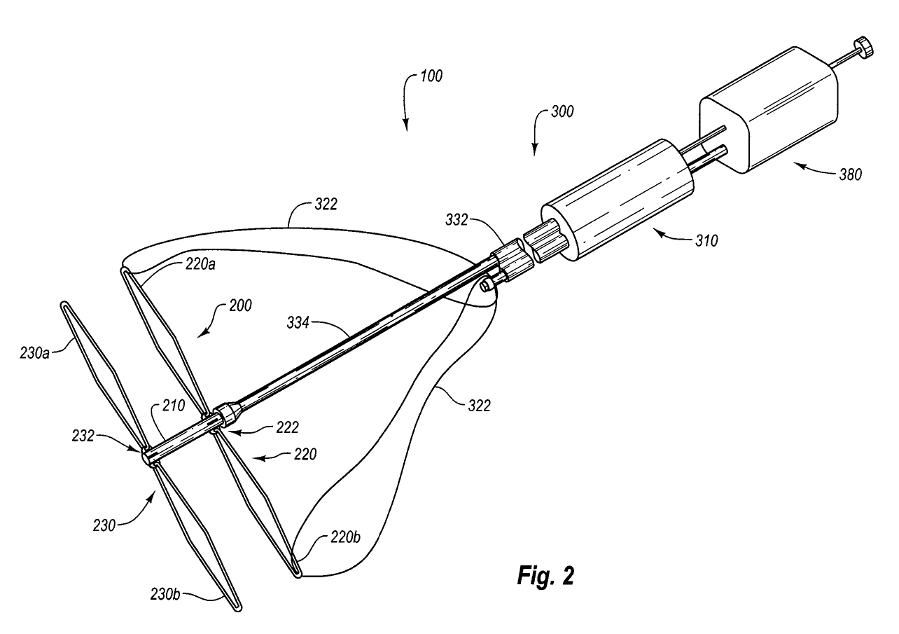

With reference now to FIG. 2, illustrated is a medical system 100 for use in reducing the size of an internal tissue opening such as a PFO, for example. In the illustrated embodiment, medical system 100 can include a PFO closure device 200 and a delivery device 300 for delivering and positioning PFO closure device 200. Delivery device 300 enables a practitioner to position a PFO closure device 200. For example, delivery device 300 can be configured to enable a practitioner to adjust and reposition PFO closure device 200 after PFO closure device 200 is positioned in a PFO, as will be described more fully hereinafter. Also, delivery device 300 can be configured to facilitate an unobstructed view of PFO closure device 200 when positioned in an opening, while maintaining connection with PFO closure device 200 in the event that repositioning is needed. Furthermore, delivery device 300 enables a practitioner to position PFO closure device 200 by utilizing conveniently positioned controls.

In the illustrated embodiment, PFO closure device 200 can include a stem 210, a right anchor 220 linked to stem 210 via a right anchor hub 222, and a left anchor 230 linked to stem 210 via a left anchor hub 232. Right and left anchors 220, 230 may include one or more arms 220a, b and 230a, b configured to engage the tissues of the heart 10. It will be understood that left anchor 230 and right anchor 220 can include more than the two arms and may have any of a variety of shapes known in the art. For example, U.S. patent application Ser. No. 11/102,095, filed Apr. 8, 2005 and incorporated herein by reference, discloses various compatible shapes and configurations for left and right anchors.

As further illustrated in FIG. 2, delivery device 300 can include a filament control system 310 connected to a handle 380. Filament control system 310 can be configured to facilitate the positioning of PFO closure device 200 in a patient. Likewise, handle 350 can be configured to work in conjunction with filament control system 310 to enable positioning and securement of PFO closure device 200 in an internal tissue opening. Filament control system 310 and handle 380 contain mechanisms which facilitate deployment and retraction of left and right anchors 230, 220. In the illustrated embodiment, delivery device 300 can be connected to PFO closure device 200, at least in part, by pusher catheter 332 via associated pusher catheter shaft 334 and by one or more filaments 322. It should be understood in light of the disclosure provided herein that for each arm of right anchor there can be a filament associated therewith, such that all arms of right anchor can be selectively retracted and deployed.

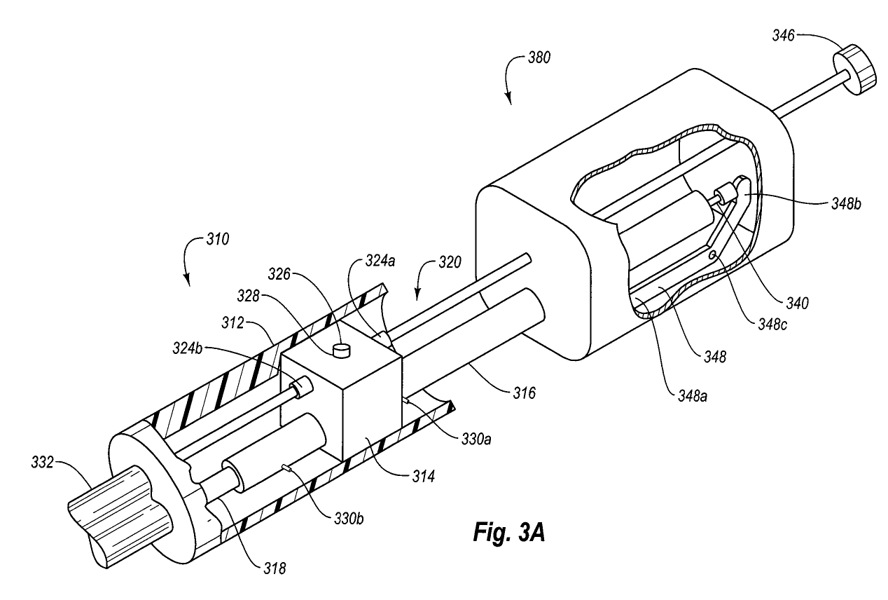

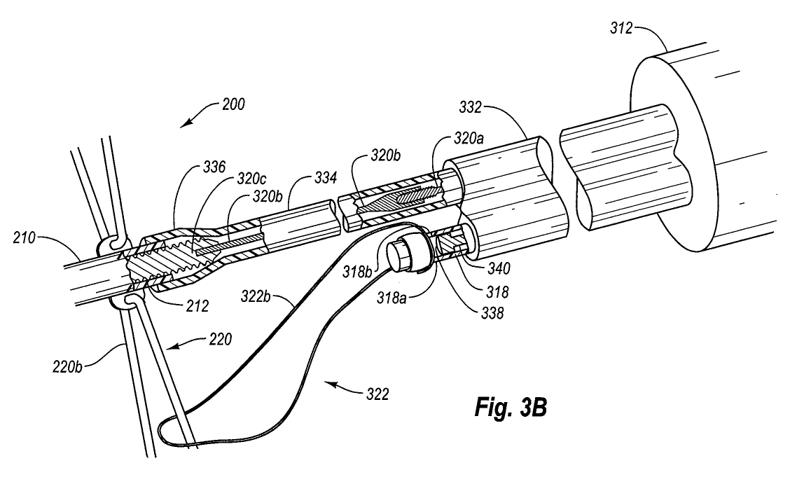

FIG. 3A is a perspective partial break-away view of filament control system 310 and handle 380 of delivery device 300. Handle 380 and filament control system 310 operate in conjunction to enable a practitioner to position PFO closure device 200 by controlling the deployment of left and right anchors 230, 220. The ability to control the position of left and right anchors 230, 220 enables a practitioner the ability to adjust and reposition PFO closure device 200.

Filament control system 310 facilitates selective deployment of right anchor 220 and selective repositioning of right anchor 220 after right anchor 220 has been deployed. As such, filament control system 310 enables right anchor 220 to be selectively deployed and selectively retracted by a practitioner using delivery device 300. Likewise, handle 380 can work in conjunction with filament control system 310 to enable a practitioner to position PFO closure device 200 inside an internal tissue opening and to remove delivery device 300 when PFO closure device 200 is appropriately positioned.

As shown in the illustrated embodiment, a tether shaft 320 can extend through handle 380 in an arrangement such that tether shaft 320 is capable of rotational movement within handle 380. A filament drive rod 316 couples to handle 380 and terminates at the proximal end within handle 380 such that a filament cutting shaft 340 can be exposed therefrom within handle 380. Coupled to handle 380 adjacent a proximal end of filament cutting shaft 340 is a filament cutting handle 348. Filament cutting handle 348 can be pivotally coupled to handle 380 such that movement of a handle portion 348a about a pivot point 348c in a direction away from filament drive rod 316 causes a portion of filament cutting handle 348 to rotate. Rotation of filament cutting handle 348 in this manner causes an engagement portion 348b to contact filament cutting shaft 340. Continued rotation of filament cutting handle 348 in this direction will cause filament cutting shaft 340 to translate and move distally through filament drive rod 316.

While handle 380 is illustrated as being substantially hollow in construction, it will be appreciated that handle 380 can be constructed to be substantially solid with recesses of sufficient size and configuration so as to allow filament cutting handle 348 and filament cutting shaft 340 to operate as described herein. Furthermore, a pin can be implemented at pivot point 348c so as to pivotally couple filament cutting handle 348 to handle 380 and to enable filament cutting handle 348 to pivot about pivot point 348c. Furthermore, it will be appreciated by one of ordinary skill in the art in view of the disclosure provided herein that filament cutting handle 348 may alternatively be positioned in housing 312, or may be integrally coupled into filament cutting shaft 340 such that filament cutting shaft 340 and filament cutting handle 348 form a single piece.

In the illustrated embodiment, filament control system 310 can include a housing 312, a shuttle block 314 positioned in housing 312 and moveable along at least a part of the length of housing 312, filament drive rod 316 and a filament operating shaft 318 coupled to filament drive rod 316. In one embodiment, housing 312 can include a cylindrical tube having an elongate hollow portion extending at least partially along the length of housing 312 and configured to house various elements of filament control system 310.

In the illustrated embodiment, filament drive rod 316 and tether shaft 320 connect housing 312 to handle 380. Tether shaft 320 extends through handle 380 and can be capable of rotational movement therein. Filament drive rod 316 can be fixed to handle 380 such that movement of handle 380 causes movement of filament drive rod 316. Collars 322a, b are coupled to tether shaft 320, each on opposite sides of shuttle block 314 such that movement of tether shaft 320 causes movement of shuttle block 314.

As shown in the illustrated embodiment, shuttle block 314 includes an aperture or opening 324 in which a pin 326 is received. Pin 326 can be utilized to link tether shaft 320 to one or more filaments 322. Filament drive rod 316 can include a first rod pin 330a and a second rod pin 330b positioned in and extending through the sidewalls of filament drive rod 330. Rod pins 330a, b are utilized in connection with safety features of delivery device 300, as will be discussed hereinafter.

FIG. 3B is a perspective view of the distal end of the delivery device 300. Further shown are partial cutouts illustrating the connection between delivery device 300 and PFO closure device 200, as well as the distal end of filament operating shaft 318. As shown in the illustrated embodiment, tether shaft 320 and filament operating shaft 318 are housed within a pusher catheter 332. Pusher catheter 332 can be coupled at its proximal end to the distal end of housing 312 and can be configured to facilitate positioning of PFO closure device 200. Furthermore, pusher catheter 332 can be configured to house tether shaft 320 and filament operating shaft 318.

In one embodiment, pusher catheter 332 can be a double-lumen catheter with tether shaft 320 being housed in a first lumen of pusher catheter 332 and filament operating shaft 318 being housed in a second lumen of pusher catheter 332. Tether shaft 320 can be capable of rotational and translational movement within pusher catheter 332. Likewise, filament operating shaft 318 can be capable of translational movement within pusher catheter 332. In this manner, a user of delivery device 300 can rotate tether shaft 320 about its central axis relative to pusher catheter 332. Also, a practitioner can move the distal end of filament operating shaft 318 closer to or further away from PFO closure device 200. The ability to move the distal end of filament operating shaft 318 closer or further away from PFO closure device 200 enables the practitioner to selectively control deployment of an atrial anchor, such as right anchor 220, of PFO closure device 200.

In the illustrated embodiment, pusher catheter 332 can be coupled to the distal end of housing 312 and can extend to substantially the distal end of delivery device 300. Pusher catheter 332 can include a pusher catheter extending shaft 334 and a pusher catheter tip 336 at the distal end of pusher catheter extending shaft 334. Pusher catheter extending shaft 334 can be configured to provide some degree of rigidity to a flexible second portion 320b of tether shaft 320 to facilitate placement of PFO closure device 200. Pusher catheter extending shaft 334 can extend from the distal end of filament operating shaft 318 and can terminate at pusher catheter tip 336.

Pusher catheter tip 336 can be configured to engage a proximal end of stem 210 of PFO closure device 200. Furthermore, pusher catheter tip 336 can be configured to be repositionable over stem 210 after pusher catheter tip 336 has been disengaged from stem 210. This can be done by a user moving tether shaft 320 in the proximal direction with respect to housing 312. With the pusher catheter 332 coupled to housing 312 and pusher catheter tip 336 coupled to pusher catheter 332, movement of tether shaft 320 in this manner would force the proximal end of stem 210 back into pusher catheter tip 336.

As shown in the illustrated embodiment, tether shaft 320 can include a first portion 320a and a second portion 320b coupled thereto. First portion 320a can include a resilient rod configured to be rotatable and provide enough stiffness to delivery device 300 so as to substantially prevent bucking of delivery device 300 as it is being used to deliver a PFO closure device 200. First portion 320a of tether shaft 320 further can include a threaded portion at the distal end which can be configured to be received within and engage internal threads of the second portion 320b of tether shaft 320. While the illustrated embodiment demonstrates that first and second portions 320a,b may be coupled through use of threads, it will be understood that a variety of types of connection means may be employed to connect first portion 320a to second portion 320b.

Second portion 320b can include a flexible rod comprising a shape memory material such as a shape memory alloy, a shape memory polymer, or the like. In one embodiment, the shape memory material can be NITINOL. Furthermore, coupled to the distal end of second portion 320b can be a threaded portion 320c. Threaded portion 320c can be configured to correspond to a set of internal threads 212 at the proximal end of stem 210. As will be appreciated, rotation of tether shaft 320 with respect to PFO closure device 200 will cause threaded portion 320c of tether shaft 320 to disengage from the internal threads 212 of stem 210. In this manner, delivery device 300 can be disengaged from PFO closure device 200 subject to connection by one or more filaments 322.

Furthermore, it will be understood by one of ordinary skill in the art in view of the disclosure provided herein that a variety of types of connection means may be employed to selectively couple tether shaft 320 to PFO closure device 200. For example, in an alternative embodiment, tether shaft 320 can be coupled to PFO closure device 200 by a hook and latch. Furthermore, it will be understood by one of ordinary skill in the art that a securing agent, such as an adhesive, can be applied between first portion 320a and second portion 320b of tether shaft 320 such that rotation of tether shaft 320 can cause disengagement of PFO closure device 200 from tether shaft 320 rather than disengagement of first portion 320a from second portion 320b. Alternatively, reverse threads can be utilized in one of the two coupling arrangements.

As shown in the illustrated embodiment, filament operating shaft 318 can be connected to arm 220b of right anchor 220 by filament 322b. In this manner, movement of filament operating shaft 318 causes movement of right anchor 220, specifically arm 220b, when filament 322b is taut. Filament operating shaft 318 can include openings 318a, b through which one or more filaments 322 pass.

Openings 318a, b in filament operating shaft 318 can be configured to facilitate severing of filaments 322 in preparation of removal of delivery device 300 from a patient. Openings 318a, b can be sized and configured to allow one or more filaments 322 to be positioned therethrough. Openings 318a, b can be positioned and located at the distal end of filament operating shaft 318. At the distal end of filament operating shaft 318 and adjacent to openings 318a, b, is a rounded portion that provides a rounded surface for filaments 322, such that when tension is induced on filaments 322, filaments 322 are not severed by a sharp outside edge of openings 318a, b.

Filament 322b can be coupled on one end to filament operating shaft 318 then pass through openings 318a, b, loop around arm 320b and then can be fixed to the delivery device 300. In this manner, as filament operating shaft 318 is moved in the distal direction with respect to housing 312, at least one end of filament 322b moves towards PFO closure device 200, thus enabling arm 220b to deploy. Likewise, as filament operating shaft 318 is moved in the proximal direction with respect to housing 312, at least one end of filament 322b moves away from PFO closure device 200, thus retracting arm 220b of right anchor 220 toward pusher catheter shaft 334.

Filament cutting shaft 340 can be housed within filament operating shaft 318 and can be capable of translational and/or rotational movement therein. In the illustrated embodiment, a filament cutting shaft can include a filament cutting portion 338 at the distal end of filament cutting shaft 340. Filament cutting portion 338 may be a sharp tip on the terminating distal end of filament cutting shaft 340 or may be a separate piece connected to the distal end of filament cutting shaft 340. Filament cutting portion 338 can be configured to sever filaments 320, which are positioned in openings 318a, b. In the illustrated embodiment, filament cutting portion 338 can include a substantially cylindrical member whose outer diameter substantially corresponds with the inner diameter of filament operating shaft 318. In this manner, as filament cutting portion 338 moves past openings 318a, b, filaments 322 are severed by a sharp leading edge of filament cutting portion 338.

It will be understood by one of ordinary skill in the art in view of the disclosure provided herein that filaments 322 can be actuated and/or severed by a variety of different configurations. For example, in one embodiment, filaments 322 can be coupled to filament cutting shaft 340, wherein filament cutting shaft can be rotated thus causing filaments 322 to be wound around filament cutting shaft 340. In this embodiment, an atrial anchor, such as a right anchor, which is looped by a filament, can be selectively moved between the deployed and retracted orientation by rotating filament cutting shaft. In an alternative embodiment, filaments 322 can be fixed to filament operating shaft 318, wherein filament operating shaft 318 can be rotated in a similar manner, thus causing filaments 322 to wind around filament operating shaft 318.

In an alternative embodiment, filaments 322 can selectively be disconnected from PFO closure device 200 by rotating filament cutting shaft 340. In this alternative embodiment, filament cutting shaft 340 can include an aperture through which one end of a filament 322 is received. Filament 322 can then be wrapped around filament cutting shaft 340 so as to secure the end of filament 322. After PFO closure device 200 has been placed, a practitioner could disconnect filament 322 from right anchor 220 by unwrapping filament 322 from filament cutting shaft 340. Filament 322 can be unwrapped by rotating filament cutting shaft 340. The atrial anchors can be manipulated and the filaments can be severed by a variety of different mechanisms and/or configurations as will be appreciated by one of ordinary skill in the art in view of the disclosure provided herein.

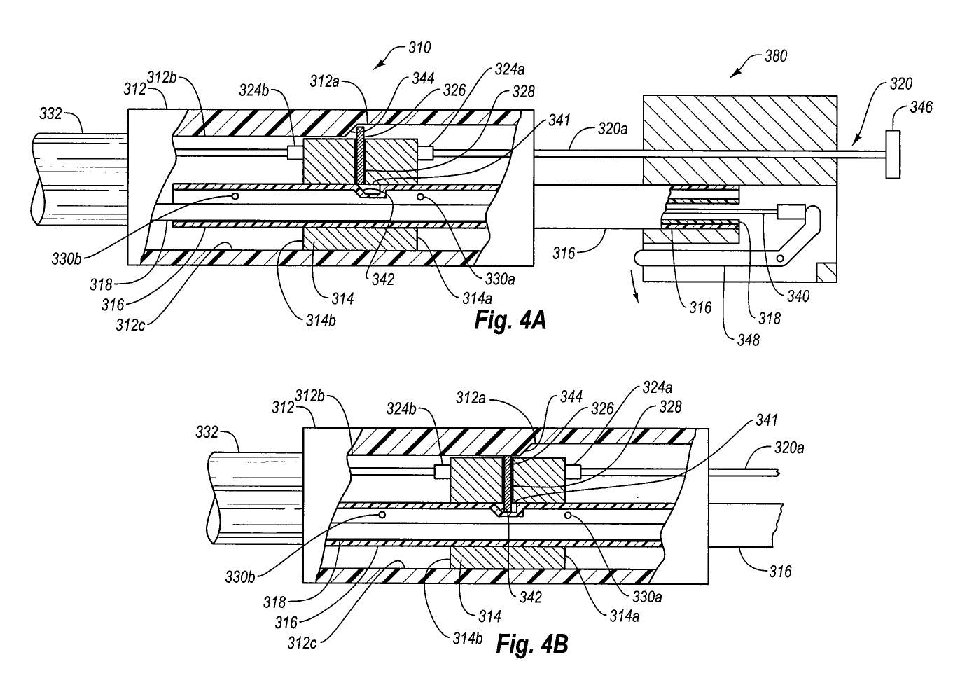

FIG. 4 is a cutaway side view of filament control system 310 and handle 380. Filament drive rod 316 can be received within shuttle block 314 and can be configured to be able to translate and/or rotate therein. Shuttle block 314 and filament drive rod 316 are configured to link tether shaft 320 to filaments 322. Filament drive rod 316 can include a recess 341 and a rod pin ramp 342 defining a portion of recess 341, and first and second rod pins 330a, b. Recess 341 can be configured to receive a portion of pin 326 therein, thus linking tether shaft 320 to one or more filaments 322. Recess 341 can be positioned in filament drive rod 316 to enable pin 326 to be forced therein as shuttle block 314 and filament drive rod 316 move in the distal direction with respect to housing 312. More specifically, recess 341 can be positioned along the length of filament drive rod 316 with respect to first rod pin 330a so as to correspond with opening 328 of shuttle block 314 when first rod pin 330a is in contact with and engages a first side 314a of shuttle block 314.

Rod pin ramp 342 can be configured to force pin 326 out of recess 341 to enable a user to retract right anchors 220 as will be discussed more fully herein. Rod pin ramp 342 defines a distal portion of recess 341 and can be of sufficient pitch so as to cause pin 326 to move upward toward a first top surface 312a of housing 312 when filament drive rod 316 is moved in the proximal direction with respect to shuttle block 314.

Filament drive rod 316 can be coupled to handle 380 such that movement of handle 380 causes movement of filament drive rod 316. In one embodiment, filament drive rod 316 can include a hollow substantially rigid shaft. First rod pin 330a can be configured and positioned in filament drive rod 316 so as to cause filament drive rod 316 to engage shuttle block 314 as filament drive rod 316 is moved in the distal direction with respect to housing 312. Furthermore, first rod pin 330a can be configured to facilitate placement of pin 326 in recess 341.

First rod pin 330a can be positioned in and can extend through the sidewalls of filament drive rod 316. First rod pin 330a can be of sufficient length such that as filament drive rod 316 is advanced through shuttle block 314, first rod pin 330a can contact first side 314a of shuttle block 314. First rod pin 330a can be positioned along the length of filament drive rod 316, such that as first rod pin 330a contacts first side 314a of shuttle block 314, opening 328 substantially aligns with recess 341 as illustrated. In this manner, as filament drive rod 316 moves in the distal direction with respect to housing 312, the top portion of pin 326 can contact a housing pin ramp 344, thus forcing pin 326 into recess 341 of filament drive rod 316.

Linking of tether shaft 320 to filaments 322 in this manner provides safety benefits. Specifically, linking of tether shaft 320 to filaments 322 enables pusher catheter tip 336 to be disengaged from stem 210 without concern that filaments 322 are manipulating the position of right anchor 220, namely inadvertently deploying and/or retracting right anchor 220.

Second rod pin 330b can be positioned in filament drive rod 316 and configured to reduce the risk that filaments 322 are prematurely severed due to a user pulling handle 380 in the proximal direction. Second pin rod 330b can be positioned in and extend through the sidewalls of filament drive rod 316. Second pin rod 330b can be positioned in filament drive rod 316 such that as filament drive rod 316 is advanced through shuttle block 314 in the proximal direction with respect to shuttle block 314, second rod pin contacts a second side 314b of shuttle block 314.

Second pin rod 330b can be positioned along the length of filament drive rod 316 so as to substantially prevent a user from inducing tension in filaments 322 sufficient to break filaments 322. For example, when right anchor 220 is completely retracted due to the tension in filaments 322, second rod pin 330b can be in contact with and engage second side 314b of shuttle block 314. In this manner, movement of filament drive rod 316 in a proximal direction with respect to housing 312 corresponds with movement of tether shaft 320 in the proximal direction with respect to housing 312, thus preventing filaments 322 from being stretched to failure.

In the illustrated embodiment, housing 312 can include a first top surface 312a, a second top surface 312b, a bottom surface 312c and a housing pin ramp 344 connecting first top surface 312a to second top surface 312b. First top surface 312a can be a surface on which pin 326 can slide when pin 326 is not within recess 341 of filament drive rod 316. Second top surface 312b can be positioned below first top surface 312a and can provide a surface on which shuttle block 314 can slide. Bottom surface 312c provides a surface on which a bottom surface of shuttle block 314 can slide. The distance between second top surface 312b and bottom surface 312c can correspond to the height of shuttle block 314, such that as pin 326 is received within recess 341 of filament drive rod 316, second top surface 312b helps to maintain pin 326 within recess 341 as shuttle block 314 moves in housing 312.

Housing pin ramp 344 can be configured to position pin 326 in recess 341 as filament drive rod 316 is advanced in the distal direction with respect to housing 312. Housing pin ramp 344 can be positioned in housing 312 so as to facilitate the linking of tether shaft 320 to one or more filaments 322 after right anchor 220 has been deployed in preparation for removal of delivery device 300 from a patient. The linking of tether shaft 320 to filaments 322 can be advantageous for safety concerns. For example, linking in this manner reduces risks to patients because the only connection between PFO closure device 200 and delivery device 300 are flexible portion 320b of tether shaft 320 and filaments 322.

It will be understood by one of ordinary skill in the art in view of the disclosure provide herein that a variety of configurations of filament control system 310 may be utilized without departing from the scope and spirit of the invention. For example, in one embodiment, tether shaft 320 can be linked to filament drive rod 316 by a linkage positioned outside of housing 312 which couples tether shaft 320 to filament drive rod 316. Alternatively, a linking assembly can be utilized so as to selectively couple tether shaft 320 to filament drive rod 316 when filament drive rod 316 and tether shaft 320 are in a particular orientation with respect to each other. Furthermore, in an alternative embodiment, filament drive rod 316 and filament operating shaft 318 comprise a single element.

Filament operating shaft 318 can be configured to facilitate movement of one or more filaments 322 to selectively deploy and retract right anchor 220. Filament operating shaft 318 can be coupled to filament drive rod 316, such that movement of filament drive rod 316 causes movement of filament operating shaft 318. In this manner, handle 380 can be linked to filament operating shaft 318, such that movement of handle 380 results in movement of filament operating shaft 318. In one embodiment, filament operating shaft 318 can include a hollow shaft extending from filament drive rod 316 to the distal portion of delivery device 300. In the illustrated embodiment, filament operating shaft 318 can be at least partially housed by filament drive rod 316 and can extend to handle 380. As a matter of clarification, in the illustrated embodiment, filament operating shaft 318 is cross-sectioned with respect to handle 380, but is not shown in cross-section with respect to filament control system 310.

Collars 324a, b are configured to link tether shaft 320 to shuttle block 314. Collars 324a, b are located on opposite sides of shuttle block 314 and are fixed to tether shaft 320. In one embodiment, collars 324a, b are compressed onto tether shaft 320 in an interference fit type arrangement. In an alternative embodiment, collars 324a, b are fixed to tether shaft 320 by an adhesive, or alternatively, by screws positioned through collars 324a, b, respectively, to tether shaft 320. Collar 324a can be positioned on the first side 314a of shuttle block 314 and collar 324b can be positioned on second side 314b of shuttle block 314. In this manner, tether shaft 320 can be capable of rotating within shuttle block 314, but translation of tether shaft 320 with respect to shuttle block 314 can be substantially prohibited.

Handle 380 can be configured to facilitate placement of PFO closure device 200. Handle 380 can be of sufficient size and shape to allow a practitioner to hold and move handle 380. Handle 380 can be connected to filament control system 310 via tether shaft 320 and filament drive rod 316. Handle 380 can be configured to enable tether shaft 320 to rotate therein and translate at least partially therethrough. Tether shaft 320 can be prevented from pulling through handle 380, at least in part, by knob 346. Knob 346 can be coupled to the proximal end of tether shaft 320. Knob 346 can be configured to facilitate removal of tether shaft 320 from PFO closure device 200. Knob 346 can be coupled to the proximal end of first portion 320a of tether shaft 320. Rotation of knob 346 causes rotation of tether shaft 320.

Knob 346 can also serve to reduce the likelihood of filaments 322 being damaged. For example, the length of tether shaft 320 can be such that as handle 380 is moved in the proximal direction, knob 346 will contact handle 380 when filaments 322 are taut and right anchor 220 is in a fully retracted orientation. In this manner, knob 346 can serve to substantially prevent filaments 322 from being damaged due to movement of handle 380.

Housed within and coupled to handle 380 can be filament drive rod 316 and filament cutting handle 348. Housed within filament drive rod 316 can be filament operating shaft 318 and filament cutting shaft 340. Filament cutting handle 348 can be the actuation mechanism utilized to sever filaments 322 upon disconnecting delivery device 300 from PFO closure device 200. Filament cutting handle 348 can be rotatably or pivotally coupled to handle 380. Filament cutting handle 348 can be of a sufficient length to actuate filament cutting shaft 340 when filament cutting handle 348 is rotated or pivoted, as discussed above. As filament cutting handle 348 is rotated in the direction shown, a portion of filament cutting handle 348 contacts filament cutting shaft 340 causing filament cutting shaft 340 to translate through filament operating shaft 318. As filament cutting shaft 340 is translated through or moves through filament operating shaft 318, filament cutting portion 338 can move to sever filaments 322 at openings 318a, b in filament operating shaft 318.

It will be appreciated by one of ordinary skill in the art in view of the disclosure provided herein that filament cutting shaft 340 can be actuated by alternative configurations. For example, in an alternative embodiment, filament cutting shaft 340 comprises a handle member coupled thereto such that movement of the handle member is the distal direction causes the filament cutting shaft 340 to slide or move within filament operating shaft 318 to sever filaments 322. In an alternative embodiment, a button or knob can be coupled to filament cutting shaft 340 through filament drive rod 316 and filament operating shaft 318. In this embodiment, a slot can be formed in filament drive rod 316 and filament operating shaft 318 sufficient to allow movement of the button or knob to in turn move filament cutting shaft 340 to sever filaments 322.

FIG. 4A illustrates pin 326 in a first position. In this orientation, tether shaft 320 is not directly linked to filaments 322. FIG. 4B illustrated pin 326 in a second position. When pin 326 is in the second position, namely, a portion of pin 326 is received in recess of 340 of filament drive rod 316, tether shaft 320 is directly linked to filaments 322. In this manner, movement of tether shaft 320 in either the distal or proximal direction with respect to housing 312 will move at least one end of filament 322 in the same direction. Further illustrated in FIG. 4B is the positioning of pin 326 relative to recess 341 of filament drive rod 316 and second top surface 312b. As illustrated, the configuration of recess 341, second top surface 312b and pin 326 can substantially maintain pin 326 in the second position when opening 328 is positioned under second top surface 312b of housing 312.

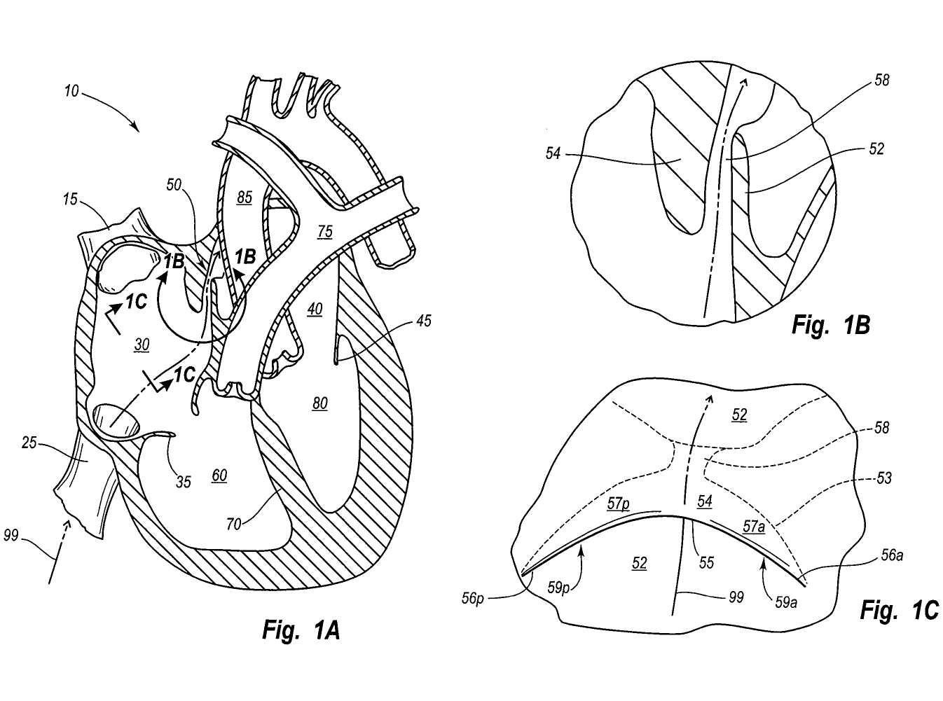

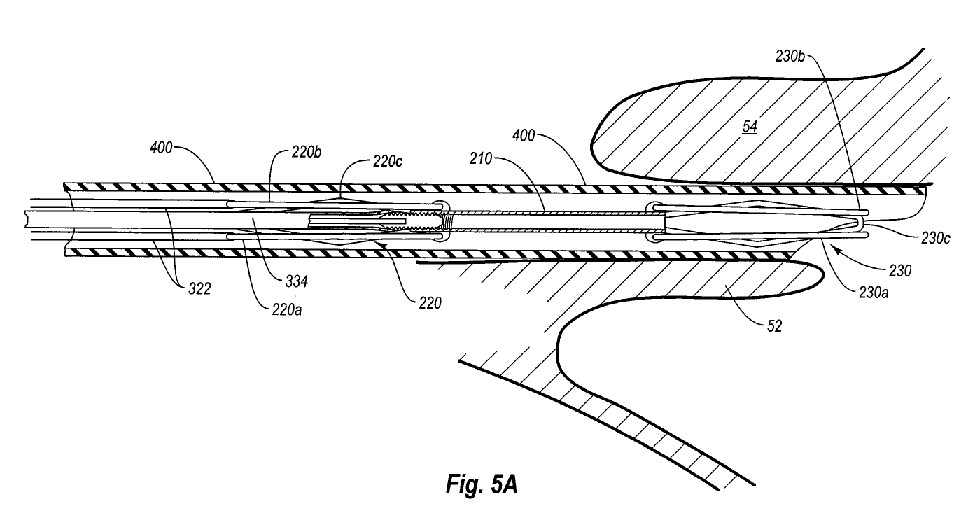

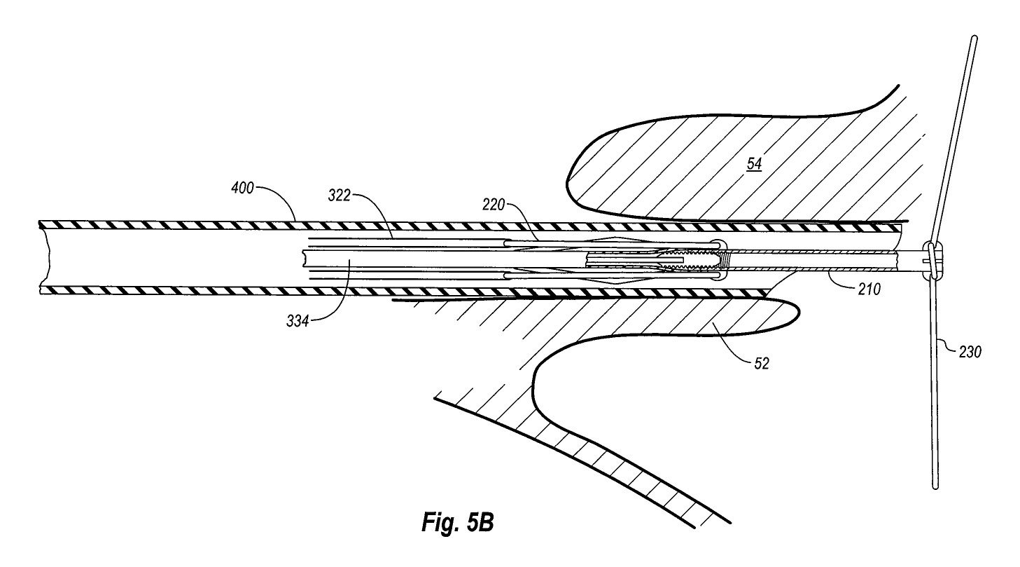

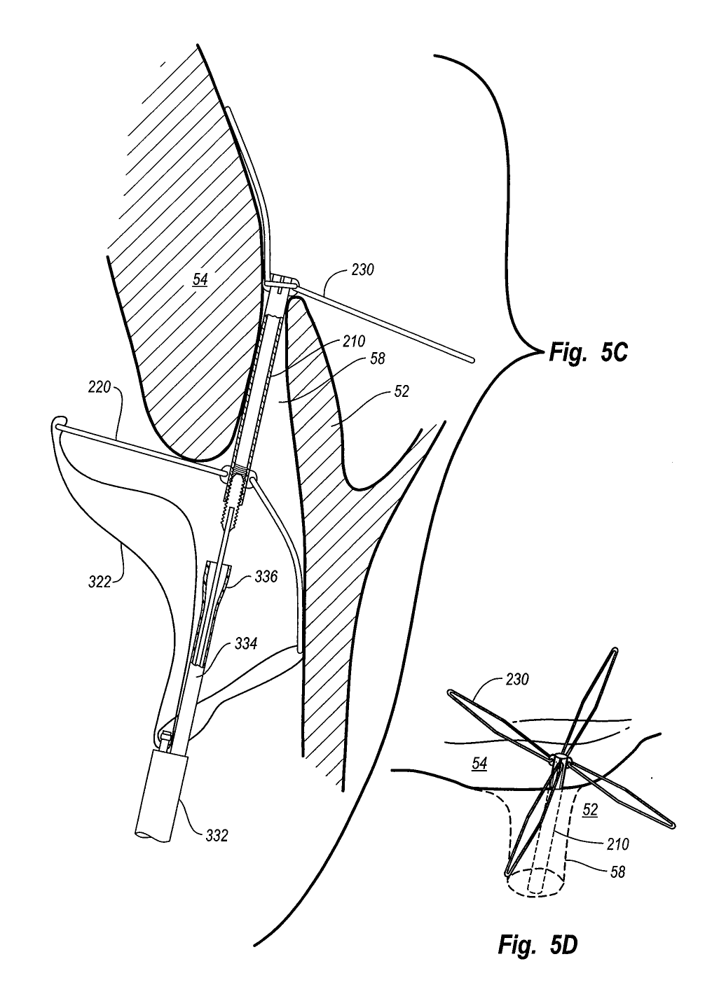

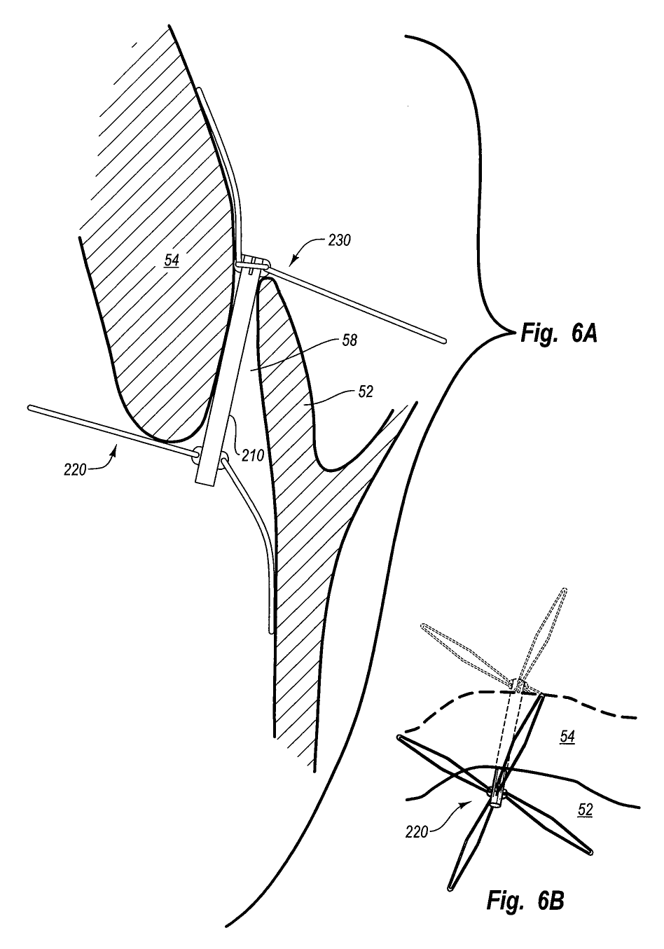

With reference to FIG. 5A-6B, one method for delivering PFO closure device 200 to PFO 50 will be described. As illustrated in FIG. 5A, a delivery sheath 400 is introduced into PFO 50 via a delivery path 99, as identified in FIGS. 1A-1C. Delivery sheath 400 is a long, somewhat flexible catheter or sheath introduced into a vein, such as femoral vein, and routed up to the right atrium of a patient's heart. The delivery sheath 400 may be tracked over a guide wire that has been advanced into the heart by a known methodology. After delivery sheath 400 is introduced into the heart via inferior vena cava 25, delivery sheath 400 is positioned at right atrium 30 in front of the inter atrial communication or PFO 50, and then through tunnel 58.

Once the distal end of delivery sheath 400 is positioned at the end of tunnel 58 as illustrated in FIG. 5A, or extends beyond tunnel 58, medical system 100 is introduced into delivery sheath 400 as shown in FIG. 5A. Specifically, PFO closure device 200 and delivery device 300 are coupled by means of stem 210 to tether shaft 320 and pusher catheter tip 336, and by filaments 322. PFO closure device 200 is introduced first into delivery sheath 400 with arms of left anchor 230 extended upward or distally and filaments taut causing right anchor 220 to be in a retracted orientation. Medical system 100 is then advanced through delivery sheath 400 until left anchor 230 extends beyond the terminating end of delivery sheath 400. In this manner, left anchor 230 will be able to return to its memory shape and will thus be deployed as shown in FIG. 5B.

A practitioner is able to utilize housing 312 to advance PFO closure device 200 through delivery sheath 400 by moving housing 312 in the distal direction. As will be appreciated, if a practitioner simply pushes on handle 380 to advance PFO closure device 200 through delivery sheath 400, pin 326 may contact housing pin ramp 344 and be inadvertently forced into recess 341.

FIG. 5A provides a cross-sectional view of PFO closure device 200 and delivery device 300 just before left anchor 220 is pushed out of delivery sheath 400 and deployed into left atrium 40. As illustrated, arms 230a-230c are extending distally such that as medical system 100 is advanced distally within delivery sheath 400, left anchors 230 deploy into left atrium 40. Furthermore, arms 220a-220c of right anchor 220 extends proximally and are held in this retracted orientation by filaments 322. FIG. 5B illustrates left anchor 220 just after deployment in left atrium 40.

FIG. 5C shows left anchor 220 being pulled proximally and positioned adjacent PFO 50. A practitioner positions PFO closure device 200 in this manner by moving housing 312 and/or handle 380 in the proximal direction. As such, a user can move left anchor 230 in the illustrated position by manipulating housing 312 and/or handle 380. Once left anchor 230 is in position, a user would move handle 380 in the distal direction with respect to housing 312, such that filament drive rod 316 and thus filament operating shaft 318 move distally with respect to housing 312. In this manner, the ends of filaments 322, which are coupled to filament operating shaft 318, are moved toward PFO closure device 200. This movement causes arms of right anchor 220 to deploy so as to extend perpendicularly from the central axis of stem 210.

In the event that the user or practitioner wishes to reposition PFO closure device 200, a user would simply move the handle 380 in the proximal direction with respect to housing 312 so as to move filament operating shaft 318 in the proximal direction. Movement in this manner causes filaments 322 to pull on arms of right anchor 220, thus moving the ends of arms of right anchor 220 in the proximal direction. In this manner, the user would be able to reposition and move the PFO closure device 200 and then again deploy right anchor 220 by moving handle 380 in the distal direction until the practitioner is satisfied with the location of PFO closure device 200.

Once right and left anchors 220, 230 are deployed and PFO closure device 200 is in a satisfactory position, the practitioner can disengage pusher catheter tip 336 from the proximal end of stem 210. This is done by moving the handle 380 in the distal direction with respect to housing 312 until pin 326 moves from the first to the second position, as shown in FIG. 4B. As will be appreciated in light of the disclosure, pin 326 is forced into recess 341 of filament drive rod 316 as filament drive rod 316 advances distally through housing 312. Furthermore, with shuttle block 314 now linking tether shaft 320 to filament drive rod 316, as handle 380 is further advanced toward housing 312, tether shaft 320 is advanced through pusher catheter 332. As tether shaft 320 is advanced distally through pusher catheter 332, it will be appreciated that tether shaft 320 will force stem 210 away from pusher catheter tip 336, thus causing disengagement. With pusher catheter tip 336 disengaged from stem 210, a practitioner is better able to view placement of PFO closure device 200 in tunnel 58.

FIG. 5C further depicts pusher catheter tip 336 being disengaged from the proximal end of stem 210, in preparation of removal of delivery device 300 from the patient. In this manner, the only connection between PFO closure device 200 and delivery device 300 are flexible filaments 320 and flexible second portion 320b of tether shaft 320. In this manner, a practitioner is able to use known methodologies to observe the positioning of PFO closure device 200 in relation to PFO 50.

Furthermore, should PFO closure device 200 require repositioning, a user would have the option of repositioning PFO closure device 200. Repositioning can be effectuated by reengaging pusher catheter tip 336 with the proximal end portion of stem 210 and inducing tension in filaments 322 by moving proximally filament operating shaft 318. Pusher catheter tip 336 can be reengaged with stem 210 by a user grasping knob 346 and moving tether shaft 320 in the proximal direction with respect to housing until pusher catheter tip 336 moves over and mates with the outer surface of stem 210. Furthermore, right anchors 220 can be retracted by a user moving handle 380 in the proximal direction with respect to housing 312, thus causing filament operating shaft 318 to translate proximally.

A user can disengage pusher catheter tip 336 from stem 210 by observing the following procedure. Once PFO closure device 200 is in position and filaments 322 are slackened, a user moves handle 380 toward housing 312 until first rod pin 330a contacts and engages first side 314a of shuttle block 314. As a user continues to move handle 380 toward housing 312, first rod pin 330a causes shuttle block 314 to move in the distal direction. As shuttle block 314 moves in the distal direction, the top portion of pin 326 will contact housing pin ramp 344, thus forcing pin 326 into recess 341 of filament drive 316. Once pin 316 is in recess 341, tether shaft 320 is linked to filaments 322 by filament drive rod 316 and filament operating shaft 318. A user would then continue to move handle 380 toward housing 312, and at the same time move housing 312 and handle 380 in the proximal direction so as to prevent right anchor 220 from being pushed into left atrium 40.

FIG. 5D illustrates the position of left anchor 230 as viewed from left atrium 40. In the illustrated embodiment, two arms of left anchor 230 are in contact with septum secundum 54 and two arms of left anchor 230 are in contact with septum primum 52.

FIG. 6A illustrates PFO closure device 200 in PFO 50 after delivery device 300 has been disconnected from PFO closure device 200. To disconnect delivery device 300 from PFO closure device 200, a user simply needs to disengage pusher catheter tip 336 from stem 210 and remove tension from filaments 322 as discussed above and then observe the following procedures. A user would rotate knob 346 so as to disengage threaded portion 320c from internal threads 212 of stem 210. Next, a user would actuate filament cutting shaft 340 by rotating filament cutting lever 348 in the direction indicated in FIG. 4A. In this manner, filament cutting portion 338 would sever filaments 322. With filaments 322 severed and threaded portion 320c of tether shaft 320 disengaged from internal threads 212 of stem 210, delivery device 300 is disconnected from PFO closure device 200. As such, delivery device 300 can then be removed from delivery sheath 400, delivery sheath 400 can then be removed from the patient, and the PFO closure device 200 is completely installed, as shown in FIGS. 6A-6B. FIG. 6B illustrates PFO closure device 200 in PFO 50 as viewed from right atrium 30.

The present invention may be embodied in other specific forms without departing from its spirit or essential characteristics. The described embodiments are to be considered in all respects only as illustrative and not restrictive. Exemplary claims have been included herein to illustrate embodiments of the invention. Although exemplary claims are presented, the invention is not limited to these claims, and the applicant reserves the right to present different or other claims in the future in view of the embodiments of the invention described herein.

What is claimed is:

1. A medical device comprising:
a tether shaft configured to be removably coupled to a closure device;
at least one filament configured to be connected to an atrial anchor of the closure device, said at least one filament configured to facilitate movement of the atrial anchor of the closure device; and
a filament operating shaft configured to be selectively linked to said tether shaft, wherein said at least one filament is connected to said filament operating shaft.

2. A medical device as recited in claim 1, wherein said tether shaft comprises a substantially rigid portion at a proximal end of said tether shaft and a flexible portion at a distal end of said tether shaft.

3. A medical device as recited in claim 1, wherein said tether shaft includes a threaded portion at a distal end of said tether shaft, wherein said threaded portion of said tether shaft is configured to correspond to a threaded portion of the closure device, such that said tether shaft can be removably coupled to the closure device via the threaded portions of said tether shaft and the closure device.

4. A medical device as recited in claim 1, further comprising:
a shuttle block linked to said tether shaft and movably coupled to said filament operating shaft, said shuttle block comprising an aperture, and
a pin positioned in said aperture of said shuttle block, such that said pin can translate in said aperture of said shuttle block.

5. A medical device as recited in claim 4, wherein said filament operating shaft includes a recess configured to receive a portion of said pin therein, such that when said aperture of said shuttle block is aligned with said recess of said operating shaft at least a portion of said pin can be positioned in said recess so as to link said tether shaft to said filament operating shaft.

6. A medical device as recited in claim 1, further comprising a housing and a catheter coupled to said housing, wherein said tether shaft is received in said housing and said catheter.

7. A medical device as recited in claim 6, wherein movement of said tether shaft in the distal direction relative to said housing causes a flexible distal portion of said tether shaft to be exposed from said catheter.

8. A medical device comprising:
a tether shaft configured to be coupled to a closure device;
at least one filament configured to be connected to an atrial anchor of the closure device; and
a filament operating shaft coupled to said at least one filament, said filament operating shaft being configured to be movable with respect to said tether shaft such that movement of said filament operating shaft with respect to said tether shaft causes movement of the atrial anchor of the closure device when the closure device is coupled to said medical device.

9. A medical device as recited in claim 8, wherein said at least one filament is connected to the atrial anchor by being fixed at one end to said filament operating shaft, then extending through an arm of the atrial anchor, and then being fixed again to filament operating shaft.

10. A medical device as recited in claim 8, wherein movement of said filament operating shaft in a distal direction with respect to said tether shaft causes the atrial anchor to move toward a deployed orientation, and movement of said filament operating shaft in a proximal direction with respect to said tether shaft causes the atrial anchor to move toward a retracted position.

11. A medical device as recited in claim 10, wherein the atrial anchor is a right atrial anchor.

12. A medical device as recited in claim 8, further comprising a handle, wherein said handle is linked to said tether shaft, and a filament drive rod coupled to said handle, wherein said filament drive rod is coupled to said filament operating shaft such that movement of said handle causes movement of said filament operating shaft.

13. A medical device as recited in claim 8, further comprising a filament cutting shaft at least partially positioned and movable within said filament operating shaft, wherein said filament cutting shaft can translate relative to said filament operating shaft.

14. A medical device as recited in claim 13, wherein said filament cutting shaft comprises a filament cutting portion configured to sever said at least one filament coupled to said filament operating shaft.

15. A medical system for use in reducing the opening of a patent foramen ovale, the system comprising:
a patent foramen ovale closure device comprising
a stem,
a left atrial anchor coupled to a distal end of said stem, and
a right atrial anchor coupled to a proximal end of said stem; and
a delivery device for delivering a patent foramen ovale closure device, said delivery device comprising
a tether shaft removably coupled to said stem of said patent foramen ovale closure device,
a filament coupled to said right atrial anchor, wherein said right atrial anchor is configured to move between a deployed and a retracted orientation by movement of said filament.

16. A system as recited in claim 15, wherein at least one of said right atrial anchor or said left atrial anchor comprises shape memory material.

17. A system as recited in claim 15, wherein said tether shaft comprises a threaded distal end corresponding with one or more internal threads of said stem, such that rotation of said tether shaft relative to said patent foramen ovale closure device enables said tether shaft to be disconnected from said patent foramen ovale closure device.

18. A system as recited in claim 15, wherein said delivery device further comprises a handle, a housing, and a filament operating shaft at least partially housed by said housing, said tether shaft and said filament operating shaft connecting said handle to said housing.

19. A system as recited in claim 18, wherein movement of said handle relative to said housing in a distal direction reduces tension in said filament thus enabling said right atrial anchor to deploy.

20. A system as recited in claim 15, wherein said delivery device further comprises a filament operating shaft, wherein at least a portion of said filament is housed within said filament operating shaft.

21. A system as recited in claim 20, wherein movement of said filament operating shaft with respect to said tether shaft in a distal direction moves said right atrial anchor into a deployed orientation and movement of said filament operating shaft with respect to said tether shaft in a proximal direction moves said right atrial anchor toward a retracted orientation, such that the patent foramen ovale closure device can be repositioned with respect to a patent foramen ovale.

22. A method of deploying a closure device in a patent foramen ovale, comprising:
providing a medical system comprising a patent foramen ovale closure device and a delivery device therefore;
inserting said patent foramen ovale closure device at least partially through a patent foramen ovale opening;
moving a filament operating shaft to selectively deploy an anchor of said patent foramen ovale closure device; and
disconnecting said patent foramen ovale closure device from said delivery device by actuating mechanisms on a handle of said delivery device.

23. A method as recited in claim 22, wherein movement of said filament operating shaft selectively deploys a right atrial anchor of said patent foramen ovale closure device.

24. A method as recited in claim 22, wherein said delivery device comprises one or more filaments coupled to said filament operating shaft, looped around a right anchor of said patent foramen ovale closure device and then coupled to said delivery device, wherein movement of said filament operating shaft with respect to said patent foramen ovale closure device in the distal direction causes said one or more filaments to slacken thereby allowing said right anchor to move from a retracted position toward a deployed position.

25. A method as recited in claim 24, further comprising moving said filament operating shaft in a proximal direction thereby causing said right anchors to move toward a retracted position.

26. A method as recited in claim 22, wherein said disconnecting said patent foramen ovale closure device comprises disengaging a pusher catheter tip from a stem of said patent foramen ovale closure device to thereby expose a flexible portion of a tether shaft.
